(19) United States
(12) Patent Application Publication
(10) Pub. No.: US 2023/0277120 A1
(43) Pub. Date: Sep. 7, 2023

(54) METHODS FOR ACCURATELY QUANTIFYING OPTICAL PROPERTIES OF SKIN IN SUBJECTS THAT HAVE VARYING SKIN TONES

(71) Applicant: The Regents of the University of California, Oakland, CA (US)

(72) Inventors: Robert Wilson, Irvine, CA (US); Wei Jin, Irvine, CA (US); Rebecca Rowland, San Anselmo, CA (US); Gordon Kennedy, Irvine, CA (US); Anthony Durkin, Irvine, CA (US); Sahil Ahuja, Irvine, CA (US)

(21) Appl. No.: 18/117,422

(22) Filed: Mar. 4, 2023

Related U.S. Application Data
(60) Provisional application No. 63/316,997, filed on Mar. 5, 2022.

(51) Int. Cl. A61B 5/00

(52) U.S. Cl. CPC A61B 5/443; A61B 5/445; A61B 5/7246; A61B 5/7221; A61B 5/0077; A61B 5/14551

(57) ABSTRACT

The disclosure provides methods for accurately quantifying optical properties of skin in subjects that have varying skin tones, and applications thereof.

### CROSS REFERENCE TO RELATED APPLICATIONS

This application claims priority under 35 U.S.C. § 119 from Provisional Application Ser. No. 63/316,997, filed Mar. 5, 2022 the disclosure of which is incorporated herein by reference.

### STATEMENT OF GOVERNMENT SUPPORT

This invention was made with Government support under Grant No. 2R01GM108634-05 awarded by the National Institute of Health, and Grant Nos. FA9550-20-1-0052 and FA9550-17-1-0193 awarded by the U.S. Air Force Office of Scientific Research. The Government has certain rights in the invention.

### TECHNICAL FIELD

The disclosure provides methods for accurately quantifying optical properties of skin in subjects that have varying skin tones, and applications thereof.

### BACKGROUND

Quantifying the optical properties of skin is critical for diagnosis, monitoring, and treatment in dermatology. For translation to the clinical setting, it is important to have technologies that are non-invasive for measuring skin optical properties in vivo. Tissue optical properties (such as the absorption coefficient and reduced scattering coefficient) can be used to quantify the concentration of major chromophores such as oxyhemoglobin, deoxyhemoglobin, and melanin, as well as tissue morphology. However, for patients with higher levels of skin pigmentation, the tissue properties measured with diffuse optics techniques (even including pulse oximetry) can be confounded by the high concentration of melanin in the epidermis. Therefore, there is a significant need to develop improved models for analyzing diffuse optics measurements in patients with darker skin to ensure that the measured optical properties in these patients are just as accurate as those measured in patients with lighter skin.

### SUMMARY

The disclosure provides iterative, layered methods for accurately quantifying optical properties of skin in subjects that have a wide range of skin pigmentations, and uses thereof The methods of the disclosure can quantify epidermal melanin concentration in a wide variety of skin pigmentations by using spatial frequency domain imaging with a layered Monte Carlo model. The methods of the disclosure are particularly suited for subjects having darker skin tones, as the methods directly corrects for the melanin content in the epidermis using an approach where one parameter at a time is varied relative to a light skin reference and fitted to the measured data from a darker skin tone.

To obtain physiologically accurate skin optical property values, the disclosure provides an iterative method utilizing a layered Monte Carlo model. For the method, it was initially assumed that μₛ′ in darker skin is the same as that in lighter skin, and the epidermal μₐ was set as the free parameter when fitting the diffuse reflectance. To test this algorithm, data was analyzed from the forearms of 6 subjects having various levels of pigmentation, at 8 visible-to-near-infrared wavelengths. The measured reflectance was compared to both the homogeneous model and our layered model to quantify fit accuracy. At 471 nm and 526 nm for patients of Fitzpatrick skin tones IV-VI (relatively dark skin), the two-layered model provided a 10-20% improvement in fit to the AC reflectance as a function of spatial frequency, compared to the homogeneous model. These improved fits yielded epidermal absorption coefficients that were notably higher than the bulk μₐ from the homogeneous model. Fitting the extracted epidermal μₐ to a melanin extinction spectrum enabled estimation of the melanin concentration in the epidermis.

In a particular embodiment, the disclosure provides a multilayered, iterative method to accurately quantify optical properties of skin in subjects that have a wide range of skin tones, comprising: (1) projecting light comprising a plurality of wavelengths and/or spatial frequencies onto a portion of a subject's skin; (2) obtaining light reflectance values from the subject's skin using an imaging or light sensing device; (3) comparing the obtained light reflectance values for the plurality of wavelengths and/or spatial frequencies with an initial simulated reflectance value to calculate a % error value, wherein the initial simulated reflectance value is generated by initializing a multi-layer Monte Carlo Simulation program with tissue optical properties defined in multiple layers of skin to simulate diffuse light reflectance for a lighter skin tone; (4) perturbing the multi-layer Monte Carlo Simulation program by adjusting the light absorption coefficient for one of the layers of skin, while keeping the light absorption coefficients for the other layers of skin constant, and keeping the light scattering coefficients for the plurality of layers constant, to generate a revised simulated reflectance value; (5) comparing the light reflectance values of step (2) with the simulated reflectance value of step (3) and the revised simulated reflectance value of step (4) to calculate a % error value, wherein if the calculated % error value meets a minimum % error value, the obtained light reflectance value is considered to be an accurate light reflectance value, and one can proceed to step (6), otherwise repeat steps (4) and (5); and (6) quantifying the optical properties of the subject's skin at each wavelength based upon the inputs of the simulation required to model the accurate light reflectance value. In another embodiment, the light projected onto the portion of the subject's skin has wavelengths from 100 nm to 2000 nm. In yet another embodiment, the light projected onto the portion of the subject's skin comprises 2 to 2000 different wavelengths. In a further embodiment, light projected onto the portion of the subject's skin comprises 2 to 3000 different spatial frequencies. In another embodiment, the imaging or light sensing device or system is selected from Spatial Frequency Domain Imaging, Temporal Frequency Domain Diffuse Optical Spectroscopy, Spatial Frequency Domain Spectroscopy, Time-Resolved Diffuse Optical Spectroscopy, Spatially-Resolved Diffuse Optical Spectroscopy, Continuous-wave near-infrared spectroscopy, Optical Coherence Tomography, pulse oximetry, dermoscopy, and colorimetry. In a certain embodiment, the imaging or light sensing device or system is Spatial Frequency Domain Imaging. In another embodiment, the subject has a skin tone that is graded type I, type II, type III, type IV, type V, or type VI on the Fitzpatrick scale. In a further embodiment, the subject has a skin tone that is graded type IV, type V, or type VI on the Fitzpatrick scale. In another embodiment, the accurate light reflectance value correlates with the concentration of a major chromophore found in the subject's skin. In yet another embodiment, the major chromophore is selected from oxyhemoglobin, deoxyhemoglobin, and melanin. In a certain embodiment, the major chromophore is melanin. In another embodiment, the multiple layers of skin comprise 2 layers of skin: a top epidermis layer, and a bottom dermis/hypodermis layer. In a further embodiment, the thickness of the top epidermis layer is set a fixed value from 30 nm to 300 μm. In yet a further embodiment, the light absorption coefficient for the epidermis layer is perturbed in the Monte Carlo Simulation program. In a certain embodiment, the multi-layer Monte Carlo Simulation is a 2-layered Monte Carlo Simulation. In a further embodiment, the optical properties of the skin being measured are selected from tissue absorption coefficient, tissue reduced scattering coefficient, and/or tissue oxygenation (StO2). In yet a further embodiment, the portion of the subject's skin comprises a skin lesion selected from a skin cancer, an ulcer, or a bum wound, and wherein the multilayered, iterative method quantifies the optical properties of the skin lesion.

In a certain embodiment, the disclosure also provides a diffuse optical technique that is used to measure tissue properties of the skin or of underlying tissues through the skin comprising a multilayered, iterative method of any one of the preceding claims. In a further embodiment, the diffuse optical technique is selected from Spatial Frequency Domain Imaging, Temporal Frequency Domain Diffuse Optical Spectroscopy, Spatial Frequency Domain Spectroscopy, Time-Resolved Diffuse Optical Spectroscopy, Spatially-Resolved Diffuse Optical Spectroscopy, Continuous-wave near-infrared spectroscopy, or Optical Coherence Tomography, pulse oximetry, dermoscopy, and colorimetry.

In a certain embodiment, the disclosure provides a multilayered, iterative method as substantially described and/or shown herein.

### DESCRIPTION OF DRAWINGS

FIG. 1 demonstrates how commercial Spatial Frequency Domain Imaging (SFDI) with a homogeneous tissue model (which is what is typically used) underestimates tissue oxygenation for subject with darker skin tones. Another reason for this disparity is that diffuse optics techniques often rely on mathematical models that assume the tissue is homogeneous. These models struggle to provide accurate values of tissue properties in patients with darker skin tones, which has a high concentration of melanin in the epidermis (so it cannot be treated as homogeneous).

FIG. 2 presents the Fitzpatrick Scale for characterizing skin tone. As typically practiced, the classification of skin tone is quite subjective/qualitative.

FIG. 3 shows how the colorimetry parameters do not directly quantify physiology. The “brightness” parameter (L*) correlates inversely with melanin concentration and melanosome size. Techniques that directly quantify melanin content of skin and separate the effects of melanin from other physiological parameters such as tissue oxygenation and morphology would be an improvement over standard colorimetry techniques.

FIG. 4 presents an overview of the SFDI is a technique, and how it has the potential to separate and quantify the absorption and scattering properties of tissue. Multiple wavelengths (e.g., 471 nm-851 nm) of light are projected onto the tissue over a wide field of view (e.g., 15 cm×20 cm) with different spatial frequencies (e.g., using sinusoidal patterns or square waves). Some of the light is absorbed by chromophores (e.g., melanin and hemoglobin) in the tissue, and some of the light is scattered back to the surface, where it is detected (e.g., with a camera or fibers). Light reflectance of different wavelength and different spatial frequencies are analyzed. By combing reflected light of different spatial frequencies, the absorption and scattering components can be separated.

FIG. 5 demonstrates that the tissue scattering coefficient (μₛ′) measured with SFDI can be inaccurate for patients with darker skin tones, due to the presence of epidermal melanin. (Top panel) Skin reduced scattering coefficient mₛ′ measured with conventional SFDI for subjects having three different skin tones (i.e., three different values of L* from skin colorimetry). Subjects having darker skin tones had artificially low values of mₛ′ at visible wavelengths (where melanin in the epidermis absorbs light). (Bottom panel) This effect was more prominent at shorter visible wavelengths (where melanin absorbs more light).

FIG. 6 demonstrates the confounding effect of pigmentation on quantifying skin tissue optical properties using Spatial Frequency Domain Imaging. The tissue scattering coefficient (ms') measured with SFDI can be inaccurate for patients with darker skin tones, due to the presence of epidermal melanin. For the ventral forearm, where there is high melanin content in subjects having darker skin tones, the measured value of mₛ′ correlates very strongly with the “lightness” parameter L*, illustrating the confounding effect of skin pigmentation on the measured mₛ′ value. Therefore, there is a continuing need to develop techniques to accurately quantify both the scattering and absorption of tissue for patients with darker skin (more and/or thicker epidermal melanin).

FIG. 7A-C presents various skin models for different skin colors. (A) Homogeneous model for lighter skin tones. (B) Homogeneous model for dark skin tones. (C) Layered model for darker skin tones.

FIG. 8 provides an exemplary embodiment of the layered perturbation method of the disclosure to characterize different concentration of melanin in different skin tone patients. Tissue scattering is assumed to be the same in both layers but absorption of epidermis is perturbed by melanin. It is assumed that the epidermis and dermis both have a “background” absorption coefficient μₐₒ and a melanin contribution Δμₐ is added to the absorption coefficient in the epidermis. For the model geometry, two slabs with thickness of top layer (epidermis) are set to a fixed value that could range from ˜10-1000 μm. As a first approximation, the scattering coefficient μₛₒ′ is assumed to be the same in both layers.

FIG. 9 displays a flowchart of the proposed layered perturbation method for modeling light transport in a layered model of skin.

FIG. 10 shows the difference between simulated reflectance and measured reflectance changes as a function of epidermis μₐ for a subject with a darker skin tone, measured at two different wavelengths.

FIG. 11 provides a comparison between the AC reflectance vs spatial frequency of the two-layered model compared with homogeneous model for subject with a dark skin tone (Type V). At the shorter wavelengths the two-layer model agrees significantly better with the measured calibrated reflectance and in the longer wavelength the layered model has similar fit to homogeneous model for subject with a darker skin tone (Type V). Average % error for all spatial frequencies for lower wavelength improved from 10-20% to <5% for subject with a darker skin tone (Type V).

FIG. 12A-D provides a model comparison of various wavelengths in different skin tone groups. (A) Difference of fitting in the lighter skin tone group using a homogeneous model. (B) Difference of fitting in the darker skin tone group using a homogeneous model. (C) Difference of fitting in the lighter skin tone group using a layered model. (D) Difference of fitting in the darker skin tone group using a layered model. The results demonstrate that the perturbation model of the disclosure provides much better fits to the measured data at shorter wavelengths (where absorption from melanin is prominent) for the subject having a darker skin tone.

FIG. 13 presents a Best-fit μₐ distribution spectrum among different skin tones at 471 nm. When the layered model is used, the measured absorption coefficient of the epidermis displays a strong negative correlation with L* (darker skin tone→higher absorption coefficient), as expected. When the homogeneous model was used, there was no apparent relationship between skin tone (degree of pigmentation) and tissue absorption, which is physiologically inaccurate because skin with higher melanin concentration should have a higher absorption coefficient (μₐ). Homogeneous mua=mμₐ from homogeneous skin model. Best-fit muaμₑₚ=mua of epidermis from layered skin model=mμₐₒ.

FIG. 14 presents a Best-fit μₐ absorption spectrum. Best-fit epidermis pa distribution at all wavelength of all research subjects. μₐ spectrum has similar line shape to melanin extinction spectrum. Top layer absorption coefficient (μₐ) (epidermis is the top layer of skin that contains melanin, which obstructs understanding of what is happening physiologically below the epidermis). The μₐ spectrum in top layer (epidermis) is calculated without any a priori assumptions about shape of spectrum, follows eumelanin extinction spectrum (dotted black curve). The result validates the assumption that melanin in epidermis is the source for the measured absorption coefficient in the top layer of our skin model.

FIG. 15 shows the measured tissue oxygenation (StO₂) from a healthy subject with a lighter skin tone (left) and a healthy subject with a darker skin tone (right), obtained using a commercial SFDI device with a homogeneous tissue model. The measured StO₂ is within a range typical of SFDI technology for the subject with a lighter skin tone, but well below a physically realistic value for the subject with a darker skin tone.

FIG. 16 demonstrates the relationship between Fitzpatrick skin type and tissue oxygenation (StO₂) measured using a commercial SFDI device with a homogeneous tissue model. For the ventral forearm (left), subjects having lighter skin (Types I-III) have similar measured StO₂ values, but subjects having darker skin (Types IV-VI) have systematically much lower measured StO₂ values. Since tissue oxygenation should not be physiologically affected by skin color, these results illustrate an inaccuracy of standard homogeneous models for tissue oximetry using diffuse optics technology. For the palm (right), which has much less melanin than the ventral forearm, subjects of all skin tones have similar StO₂ values. This result serves as a “negative control”, suggesting that for tissues where melanin is not present, StO₂ measurements are similar for all subjects, but for tissues where melanin is present, StO₂ measurements can be significantly confounded by melanin.

FIG. 17 demonstrates the relationship between skin “lightness” parameter (L*), measured via colorimetry, and measured tissue oxygenation (StO₂) obtained using a commercial SFDI device with a homogeneous tissue model utilizing ventral forearm measurements on 15 healthy subjects of wide-ranging skin colors. There is a strong linear correlation between the measured values of L* and StO₂: subjects having lighter skin (higher L*) have systematically higher measured values of StO₂, even though there is no reason to expect that tissue oxygenation should be physiologically related to skin tone. Therefore, these results indicate a significant bias in accuracy of the commercial SFDI device toward patients with lighter skin tones when the homogeneous tissue model is used (i.e., the device with the homogeneous model is far more accurate for subjects having lighter skin tones than for subjects having darker skin tones).

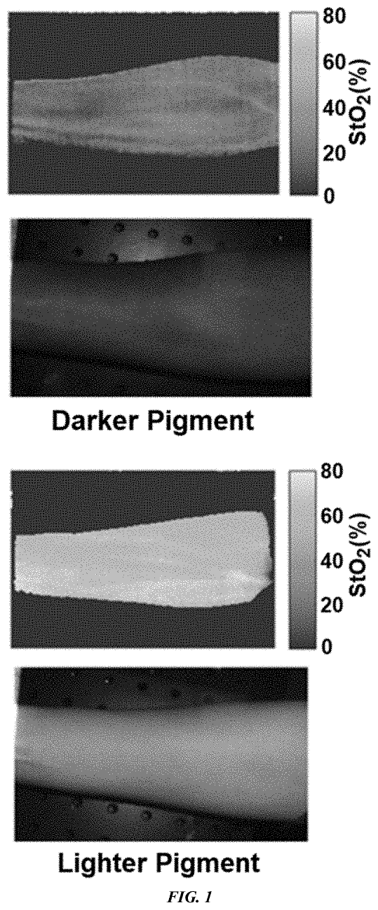

### DETAILED DESCRIPTION

As used herein and in the appended claims, the singular forms “a,” “an,” and “the” include plural referents unless the context clearly dictates otherwise. Thus, for example, reference to “a skin layer” includes a plurality of said skin layers and reference to “the image” includes reference to one or more images and equivalents thereof known to those skilled in the art, and so forth.

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated by reference in full for the purpose of describing and disclosing methodologies that might be used in connection with the description herein. The publications are provided solely for their disclosure prior to the filing date of the present application. Nothing herein is to be construed as an admission that the inventors are not entitled to antedate such disclosure by virtue of prior disclosure. Moreover, with respect to any term that is presented in one or more publications that is similar to, or identical with, a term that has been expressly defined in this disclosure, the definition of the term as expressly provided in this disclosure will control in all respects.

For purposes of this disclosure, “light” is not necessarily limited to the electromagnetic radiation that can be perceived by the human eye (i.e., visible light), but can include electromagnetic radiation from other regions of the electromagnetic spectrum such as the near ultraviolent region, near infrared region, and mid infrared region. In a particular embodiment, “light” refers to visible light. In regards to “light” comprising a plurality of wavelengths and/or spatial frequencies, such light can arise from noncoherent light sources, such as diodes, lamps, and bulbs, or can come from a plurality of coherent light sources, such as lasers, that produce light that differ by wavelength and/or spatial frequency. In a particular embodiment, “light” comprising a plurality of wavelengths and/or spatial frequencies is generated by one or more noncoherent light sources.

For purpose of this disclosure, “portion of a subject's skin” refers to an area found on a region of a subject's body. Examples of regions of a subject's body include, but is not limited to, limbs, torso, neck, face, or legs of a subject. In a particular embodiment, “portion of a subject's skin” does not include the palms, and/or soles of the feet. In another embodiment, “portion of a subject's skin” refers to an area found on the subject's forearm.

For purposes of this disclosure, “melanin” refers to all forms of melanin found in the human skin, which would include eumelanin, pheomelanin, and neuromelanin. In a particular embodiment, “melanin” refers to eumelanin.

Human skin color ranges from the darkest brown to the lightest hues. Differences in skin color among individuals is caused by variation in pigmentation, which is the result of genetics (inherited from one's biological parents and or individual gene alleles), exposure to the sun, natural and sexual selection, or all of these. Differences across populations evolved through natural selection or sexual selection, because of social norms and differences in environment, as well as regulations of the biochemical effects of ultraviolet radiation penetrating the skin.

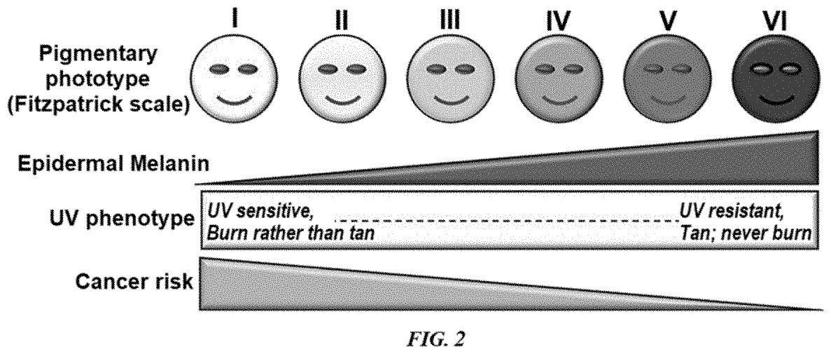

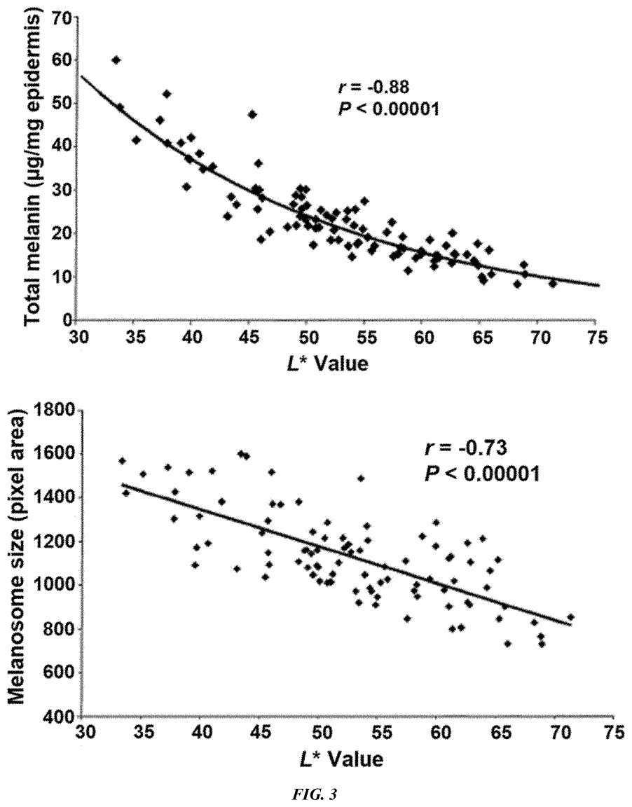

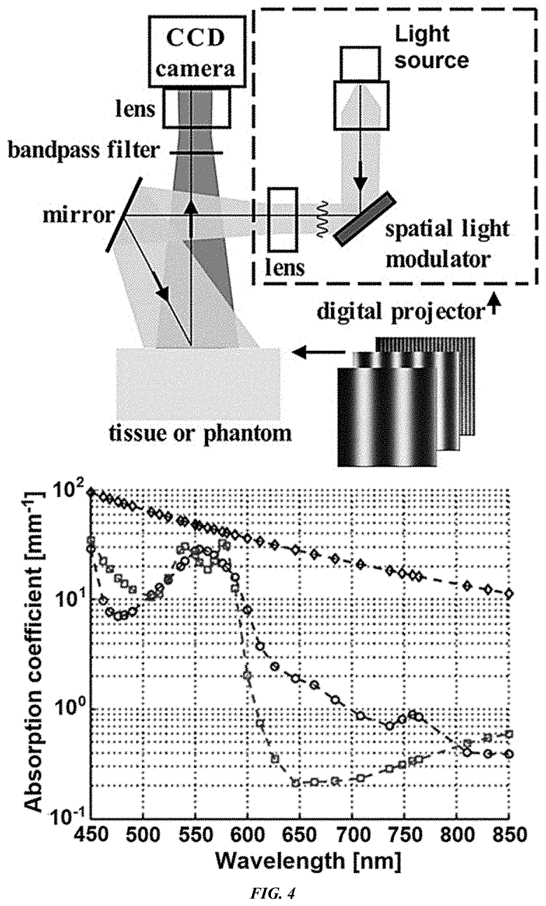

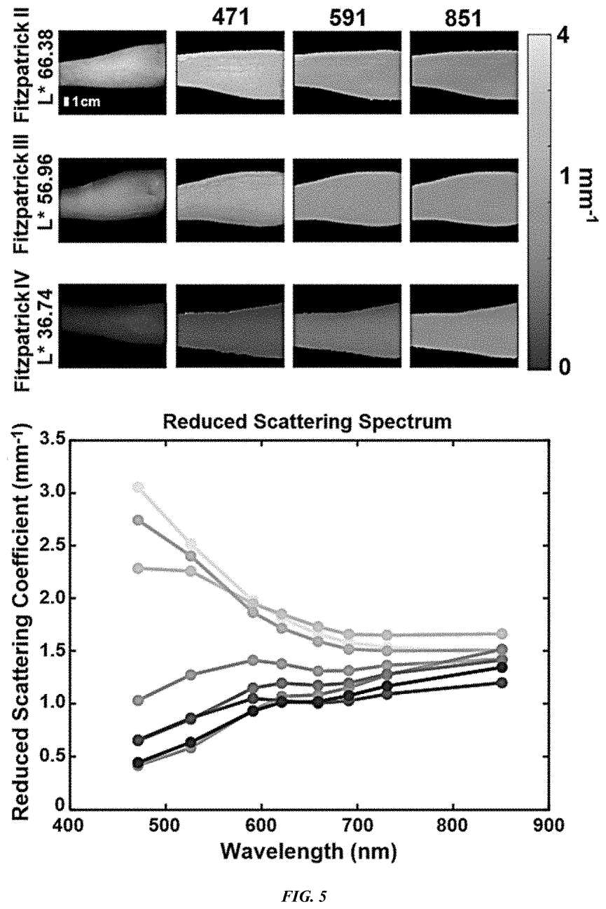

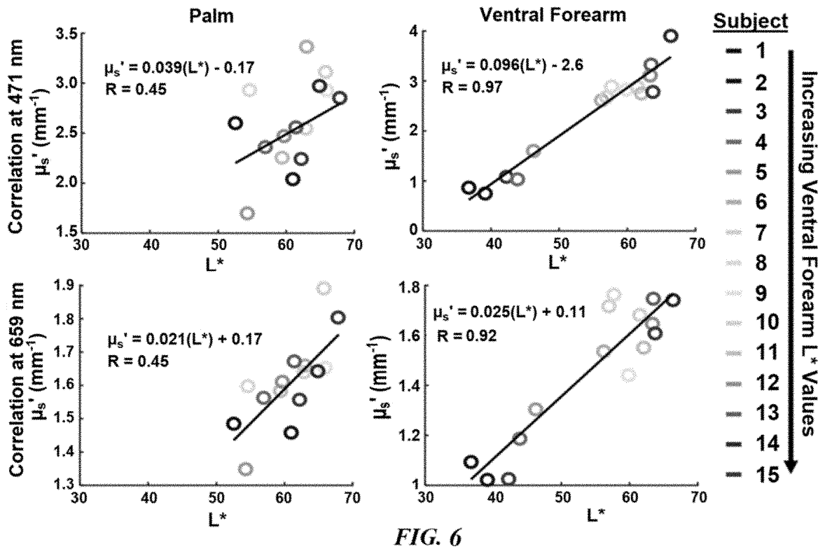

Various classification schemes have been developed to classify skin color into defined groups, such as the Von Luschan's chromatic scale (VLS) and the Fitzpatrick scale. VLS was used extensively throughout the first half of the 20th century in race studies and anthropometry. Unfortunately, the results were inconsistent: in many instances, different investigators would give different readings of the same person. As such, VLS was largely abandoned by the early 1950. The Fitzpatrick scale (see FIG. 2) remains a recognized tool for dermatological research into human skin pigmentation. It was initially developed on the basis of skin color to measure the correct dose of UVA for PUVA therapy, and when the initial testing based only on hair and eye color resulted in too high UVA doses for some, it was altered to be based on the patient's reports of how their skin responds to the sun; it was also extended to a wider range of skin types. The following table shows the six categories of the Fitzpatrick scale in relation to the 36 categories of the older VLS:

| Type | Also called | Sunburning | Tanning Behavior | VLS |
|---|---|---|---|---|
| I | Light, pale white | Always | Never | 0-6 |
| II | White, fair | Usually | Minimally | 7-13 |
| III | Medium white to light brown | Sometimes | Uniformly | 14-20 |
| IV | Olive, moderate brown | Rarely | Easily | 21-27 |
| V | Brown, dark brown | Very rarely | Very easily | 28-34 |
| VI | Very dark brown to black | Never | Rarely | 35-36 |

The actual skin color of different humans is affected by many substances, although the single most important substance is the pigment melanin. Melanin is produced within the skin in cells called melanocytes and it is the main determinant of the skin color of darker-skin humans. The skin color of people with light skin is determined mainly by the bluish-white connective tissue under the dermis and by the hemoglobin circulating in the veins of the dermis. The red color underlying the skin becomes more visible, especially in the face, when, as consequence of physical exercise or sexual arousal, or the stimulation of the nervous system (anger, embarrassment), arterioles dilate. Color is not entirely uniform across an individual's skin; for example, the skin of the palm and the sole is lighter than most other skin, and this is especially noticeable in darker-skinned people.

Melanin is produced by cells called melanocytes in a process called melanogenesis. Melanin is made within small membrane—bound packages called melanosomes. As they become full of melanin, they move into the slender arms of melanocytes, from where they are transferred to the keratinocytes. Under normal conditions, melanosomes cover the upper part of the keratinocytes and protect them from genetic damage. One melanocyte supplies melanin to thirty-six keratinocytes according to signals from the keratinocytes. They also regulate melanin production and replication of melanocytes. People have different skin colors mainly because their melanocytes produce different amount and kinds of melanin.

The genetic mechanism behind human skin color is mainly regulated by the enzyme tyrosinase, which creates the color of the skin, eyes, and hair shades. Differences in skin color are also attributed to differences in size and distribution of melanosomes in the skin. Melanocytes produce two types of melanin. The most common form of biological melanin is eumelanin, a brown-black polymer of dihydroxyindole carboxylic acids, and their reduced forms. Most are derived from the amino acid tyrosine. Eumelanin is found in hair, areola, and skin, and the hair colors gray, black, blond, and brown. In humans, it is more abundant in people with dark skin. Pheomelanin, a pink to red hue is found in particularly large quantities in red hair, the lips, nipples, glans of the penis, and vagina. Differences in human skin color can largely be attributed to the levels of eumelanin pigmentation. Lighter skin tones (e.g., Fitzpatrick Scale I, II, or III) have lower levels of eumelanin pigmentation than darker skin tones (e.g., Fitzpatrick Scale IV, V, or VI). Environmental factors, namely the levels of UV radiation, have been attributed to the evolution of skin color. The higher the levels of UV radiation, the greater the levels of eumelanin pigmentation. Eumelanin pigmentation protects tissues and DNA from radiation damage by UV light. While lower levels of eumelanin pigmentation is disadvantageous in environments with higher UV radiation (e.g., high sunlight environments), it is advantageous in lower UV radiation environments (e.g., low sunlight environments), due to the increased synthesis of vitamin D. Vitamin D production in the skin begins when UV radiation penetrates the skin and interacts with a cholesterol-like molecule produce pre-vitamin D₃. This reaction only occurs in the presence of medium length UVR, UVB. The farther a place is from the equator, the less UVB is received, and the potential to produce of vitamin D is diminished.

While skin tones can be subjectively classified by using schemes like the Fitzpatrick scale, objective characterization of skin features, including melanin and hemoglobin concentrations, the depth and diameter of blood vessels, the depth of pigmented skin lesions, the maturity and depth of bruises, and keratin fiber arrangements, are determined by measuring the optical properties of skin. Methods for measuring the optical properties of skin have proved invaluable for the advancement of skin laser treatments and photodynamic therapy, and have contributed to further advances in the diagnosis of cancerous and noncancerous skin lesions.

Optical properties of skin can be largely be attributed to absorption and scattering effects of light by various components in the layers of skin (e.g., dermal layer, epidermal layer, and hypodermal layer). Hemoglobin is the dominant absorber of light in the dermal layer of skin. Normal adult hemoglobin (Hb A) is a protein consisting four polypeptide chains, each of which is bound to a heme. The heme in Hb A is named iron-photoporphyrin IX and is responsible for the majority of light absorption in blood. The free-electron molecular-orbital model describes this absorption. as an excitation of loosely bound “unsaturation electrons” or “π-electrons” of the heme. Within the visible region, Hb A contains three distinctive peaks. The dominant peak is in the blue region of the spectrum and is referred to as the Soret peak or Soret band. Two further peaks can be distinguished in the green-yellow region, between 500 and 600 nm, that in combination with the Soret band cause Hb A to appear red. These are known as the α and β bands, or collectively as the Q-band, and have intensities of around 1% to 2% of the Soret band. The excitation levels of π-electrons vary, and therefore the positions and intensities of these bands vary with the ligand state of the heme.

Melanins are ordinarily contained within the epidermis and produce an absorption spectrum that gradually decreases from the ultraviolet (UV) to the infrared (IR) regions. In contrast to hemoglobin, the variation and complexity of melanins means that their detailed structures are not yet fully understood, despite intense research over the last five decades, and this broadband absorbance spectrum is still a topic of scientific debate. At present, the scientific consensus appears to gravitate towards a chemical disorder model. This model proposes that melanins consist of a collection of oligomers or polymers in various forms arranged in a disordered manner. This results in a number of absorption peaks that combine to create a broadband absorbance effect.

Further absorption of light may be attributed to chromophores, such as bilirubin and carotene, lipids, and other structures, including cell nuclei and filamentous proteins. Although the individual contributions from these secondary chromophores may be considered separately, most simulations group them into a single overarching value.

Despite its abundance in all tissues, water is not a significant absorber of light in the visible region, although its contribution has been considered when simulating skin color.

As well as absorption, scattering contributes significantly to the appearance of skin. Scattering describes a change in the direction, polarization or phase of light and is commonly portrayed as either a surface effect (such as reflection or refraction) or as an interaction with a small region whose optical properties differ from its surroundings (particulate scatter).

The primary sources of particulate scatter within the skin are filamentous proteins. Keratins are the filamentous proteins of the epidermis and form this layer's major constituent, whereas collagen is the principal filamentous protein of the dermis and occupies approximately 18% to 30% of its volume. Further scatter is attributed to melanosomes in the epidermis, cell nuclei, cell walls and many other structures in the skin that occur in smaller numbers.

The volume fraction of melanosomes in the epidermis varies typically from 1% in pale skin to 5% in darker skin. However, despite their low numbers relative to keratins, melanosomes are approximately 10 times the diameter of the largest keratin structures in the epidermis and possess a greater refractive index (and therefore a greater difference in refractive index at their interface with skin). Melanin has been shown to contribute significantly to the degree of scatter within the epidermis. As well as the volume fraction, the distribution and size of melanin structures in the epidermis also vary with skin type. Thus, the total amount of scatter that occurs as a result of melanin in the epidermis can vary substantially between individuals, although this is not always taken into account when simulating the effects of varying melanin concentration on skin color, or when simulating laser treatments, for example. It has been found that the subjects with darker skin tones have different optical measurements than subjects with lighter skin tones (see Svaasand et al., Lasers Med. Sci. 10(1): 55-65 (1995). The tissue absorption coefficients, and the tissue reduced scattering coefficients, however, have been largely established from subjects with lighter skin tones giving rise to bias in readings, such as overestimating arterial oxygen saturation during hypoxia in dark-skinned individuals (see Bickler et al., Anesthesiology 102(4): 715-9 (2005).

Being able to accurately quantify the optical properties of skin is critical for diagnosis, monitoring, and treatment in dermatology. For translation to the clinical setting, it is important to have technologies that are non-invasive for measuring skin optical properties in vivo. Tissue optical properties (such as the absorption coefficient and reduced scattering coefficient) can be used to quantify the concentration of major chromophores such as oxyhemoglobin, deoxyhemoglobin, and melanin, as well as tissue morphology. Currently, existing optical methods for measuring optical properties of biological tissues can be divided into direct and indirect measurement methods. The direct method is advantageous because it can use a simpler mathematical model (e.g., Beer-Lambert Law) and simpler data processing algorithm. However, this kind of method is limited in the specific types of samples that it can be used for, with strict conditions (e.g., samples must be thin) and can be confounded by the influence of stray light outside and reflection from the experimental device, such as the cuvette. In contrast, indirect methods can be performed on intact samples (including in vivo samples) nondestructively but need sophisticated instrumentation and complex mathematical models Recent studies have been mainly focused on indirect methods for estimating optical properties, because they are applicable to a wide range of biological materials without the need for sample preparations. Some commonly used optical methods for measuring tissue optical properties, including collimated transmittance, integrating sphere (IS), time-domain (TR), frequency-domain (FD), spatially resolved (SR), and spatial-frequency domain imaging (SFDI). Reflectance and/or transmittance were first measured by these techniques, and then the optical properties (i.e., absorption coefficient μa, and reduced scattering coefficient μs′) were estimated by using the inverse parameter estimation algorithms based on light transfer models. During the past years, these optical techniques have been widely used for measuring optical properties of different biological materials, such as human skin, brain, and tumor tissues.

SFDI, as an emerging optical imaging technique, is capable of measuring the tissue optical properties in a wide-field area on a pixel-by-pixel basis. Compared to other methods listed above (i.e., IS, TR, FD, and SR), SFDI employs spatially modulated area lighting, instead of point lighting, for illuminating the turbid materials, and thus 2-D and even 3-D optical property mappings can be achieved through single measurement. In the SFDI technique, special patterns of 2-D illumination, usually sinusoidal patterns, with different spatial frequencies are projected onto the surface of a target sample, and the remitted diffuse reflectance is captured by using an imaging device (e.g., high-performance camera). Demodulation algorithms, such as three-phase demodulation, Gram—Schmidt orthonormalization, and spiral phase transform, are then applied to obtain the direct component (DC) image and alternating component (AC) image. Tissue optical properties can be finally determined by fitting the AC image based on inverse parameter estimations. Biological tissue acts as a low-pass filter, thus low-frequency lighting is more sensitive to absorption, while high-frequency component performs more effects on scattering. Therefore, the SFDI technique provides potential for decoupling the absorption property from scattering property of biological tissues.

Owing to the capabilities of wide-field imaging, depth- and resolution-varying characterizing for biological tissues, SFDI has witnessed great progress in measuring optical properties. The estimated optical property values and/or mappings provide valuable information for disease diagnosis, evaluation, and monitoring in biomedical domain, as well as quality assessment in the food and agricultural engineering domain.

SFDI techniques have shown significant potential for characterizing static and dynamic properties of skin. However, for patients with higher levels of skin pigmentation, the tissue properties measured with SFDI can be confounded by the high concentration of melanin in the epidermis, as seen with subjects with darker skin tones. It has been shown herein that when measuring skin, bulk μₐ and μₛ′ from a homogeneous light transport model, the actual properties of individual skin layers, especially for highly pigmented skin, can be highly inaccurate. Accordingly, there is need to develop improve methods for using SFDI, and other optical measuring techniques for characterizing skin and other tissues, which can provide more accurate results, irrespective of the levels of skin pigmentation.

The disclosure provides innovative methods for accurately quantifying optical properties of skin in subjects that have a wide range of skin pigmentations, and uses thereof. In particular, the methods of the disclosure utilize Spatial Frequency Domain Imaging (SFDI). Current methods with SFDI, however, are confounded by the high concentrations of melanin found in the epidermis of patients having darker skin tones. To overcome the foregoing limitations with existing SFDI methods, the disclosure provides a novel, iterative, layered method for quantifying optical properties of skin in subjects that have a wide range of skin pigmentations.

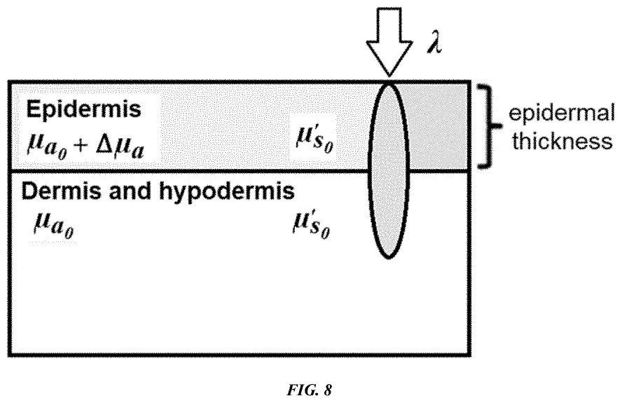

An exemplary embodiment of an iterative, layered method of the disclosure to characterize different concentration of melanin in different skin type patients is shown in FIG. 8. As shown, tissue scattering is assumed to be the same in both layers but absorption of epidermis is perturbed by melanin. It is assumed that the epidermis and dermis both have a “background” absorption coefficient μₐₒ and a melanin contribution Δμₐ is added to the absorption coefficient in the epidermis. For the model geometry, two slabs with thickness of top layer (epidermis) are set to a fixed value that could range from ˜10-1000 μm. As a first approximation, the scattering coefficient μₛₒ′ is assumed to be the same in both layers. Using a computational Monte Carlo model of photon propagation in tissue, a “library” of simulated backscattered diffuse reflectance curves Rd(λ, fₓ) are created as a function of the wavelength λ and spatial frequency fₓ of the input light for skin models I and II of the Fitzpatrick scale. For the first iteration, the “background” absorption and scattering coefficients (μₐₒ, μₛₒ′) are taken from SFDI measurements of subjects having light skin tones, using a homogeneous skin model (since this should be accurate for subjects having low melanin content). The epidermal absorption coefficient (μₐₒ and Δμₐ) is the only coefficient that is being perturbed in the Monte Carlo model to generate a simulated reflectance value. The simulated reflectance value is then compared with measured reflectance data (from skin) to find the value of Δμₐ that provides the reflectance curve with the best fit to the measured data to generate an accurate reflectance value.

As shown in the Examples presented herein, the iterative, layered methods disclosed herein notably improved current Spatial Frequency Domain Imaging technology to quantify the optical properties of dark skin more accurately. It is further envisaged that the iterative, layered methods disclosed herein can also be used with other imaging techniques, including but not limited to, Temporal Frequency Domain Diffuse Optical Spectroscopy (FD-DOS), Spatial Frequency Domain Spectroscopy (SFDS), Time-Resolved Diffuse Optical Spectroscopy (FD-DOS), Spatially-Resolved Diffuse Optical Spectroscopy, Continuous-wave near-infrared spectroscopy (CW-NIRS), or Optical Coherence Tomography. Moreover, the iterative, layered methods disclosed herein can be modified for High-Frequency Ultrasound in order to estimate thickness of top layer to use as an a priori fixed value during the fitting procedure to solve for Δμₐ.

As shown in the Examples presented herein, the iterative, layered methods disclosed herein using on a 2-layer Monte Carlo method was used to evaluate the simulation of reflectance. The results showed that after the first perturbation of epidermal μₐ, fitting of the measured reflectance is improved by 10-20% comparing to typical homogeneous simulation results for darker skin type subjects. The extracted best fit epidermal μₐ also has a very high correlation with skin colorimetry data, which indicates a new way to calculate epidermal melanin concentration. It is further envisaged that the iterative, layered methods disclosed herein can used for additional ‘higher-order’ perturbation terms in addition to epidermal μₐ including, but not limited to, perturbations to the scattering coefficient in each layer, perturbations to the epidermal thickness, perturbations to the bottom layer absorption coefficient, perturbations to tissue geometry, evaluating an irregularly-shaped interface between epidermis and dermis, and splitting dermis and hypodermis into multiple distinct layers with different optical properties. Accordingly, while the exemplary embodiments and examples provided herein are directed to a 2-layer skin model (epidermis and dermis and hypodermis), it should be understood that the techniques and methods of the disclosure are not limited to skin or to 2-layered models, or to perturbing only the epidermal μₐ coefficient.

Due to the high absorption effect of melanin in the tissues of individuals with darker skin tones, a more accurate characterization of skin optical properties is needed. As shown in the Examples herein, the iterative, layered methods of the disclosure dramatically improved the accuracy of measuring tissue properties in patients with darker skin tones. As such, quantitative diagnostic and monitoring techniques can be developed that are equally accurate across patients of all skin tones, helping to overcome health care disparities among patients with different races and skin tones.

Many applications using the iterative, layered methods of the disclosure can be envisaged, including: characterizing skin lesions for detection of cancer; monitoring of tissue oxygenation changes during pressure ulcer formation; quantifying healing of burn wounds; improving pulse oximetry in subjects having darker skin tones; and monitoring of diabetic foot ulcers.

Burn wounds are a common afflictions that usually causes damage to the skin, mucous membrane, subcutaneous and submucosal tissues, and even some complications. Accurate detection of burn location and severity is critical for determining the scheme for the treatment and recovery. SFDI has been applied for surface and subsurface burn detection, burn severity, and healing of burn wounds (see Nguyen et al., J. Burn Care Res 34: 44-50 (2013); Kennedy et al., Photonics Dermatol. Plast. Surg. 108510 (2019); Ponticorvo et al., Burns 45: 450-460 (2019); Rowland et al., Photonics Dermatol. Plast. Surg. 1085109 (2019); Ponticorvo et al., Biomed. Opt. Express 5: 3467-3481 (2014); Poon et al., Toxicol. Vitr. 52: 251-254 (2018); Nguyen et al., J. Biomed. Opt. 18: 066010 (2013); and Burmeister et al., Burns 41: 1242-1252 (2015). As the studies presented herein indicate, the iterative, layered methods of the disclosure provided non-biased results irrespective of the subject's skin tone when SFDI was utilized. As such, the methods of the disclosure would be better able to quantify burn severity, burn detection and healing of burns wounds more accurately, especially in subjects that have darker skin tones, when using SFDI.

Pulse oximetry has been found to be biased toward subjects with lighter skin tones, by providing more accurate results for tissue oxygenation levels for subjects with lighter skin tones than for subjects with much darker skin tones (see Fawzy et al., JAMA Intern. Med. 182(7): 730-738 (2022). As the studies presented herein indicate, the iterative, layered algorithms of the disclosure provide non-biased results even if the subject has a darker skin tone. Accordingly, applying the iterative, layered algorithms of the disclosure with oximeters could provide for more accurate blood oxygenation results for subjects with darker skin tones.

The SFDI technique was employed for imaging skin cancer lesions (see Saager et al., Rev. Sci. Instrum. 88: 094302 (2017)). As the studies presented herein indicate, the iterative, layered methods of the disclosure provided non-bias results irrespective of the subject's skin tone when SFDI was utilized. As such, the methods of the disclosure could more accurately characterize skin cancers, especially pigmented lesions and lesions in subjects that have darker skin tones, when using SFDI.

Melanin content and oxygen saturation can reflect the skin health status and provide much valuable information in detecting skin diseases, such as port wine stain (PWS), actinic keratosis (AK), and pressure ulcers. SFDI is advantageous in measuring these indices by extracting and mapping tissue optical properties, which can be used to evaluate the skin tissue (see Yafi et al., Lasers Surg. Med 49: 827-834 (2017). As the studies presented herein indicate, the iterative, layered methods of the disclosure provided non-biased results irrespective of the subject's skin tone when SFDI was utilized, including for StO₂. As such, the methods of the disclosure would more accurately characterize skin lesion (e.g., diabetic foot ulcers), especially in subjects that have darker skin tones, when using SFDI. Further, the monitoring of tissue oxygenation changes during pressure ulcer formation by SFDI can be more accurately performed by carrying out the iterative, layered methods of the disclosure for subjects with varying skin tones.

Additionally, the iterative, layered methods of the disclosure can be carried out using various diffuse optical techniques to measure tissue properties of the skin or of underlying tissues through the skin, including, but not limited to, pulse oximetry, dermoscopy, and colorimetry.

The disclosure further provides that the methods described herein can be further defined by the following aspects (aspects 1 to 17):

1. A multilayered, iterative method to accurately quantify optical properties of skin in subjects that have a wide range of skin tones, comprising:
(1) projecting light comprising a plurality of wavelengths and/or spatial frequencies onto a portion of a subject's skin;
(2) obtaining light reflectance values from the subject's skin using an imaging or light sensing device or system;
(3) comparing the obtained light reflectance values for the plurality of wavelengths and/or spatial frequencies with an initial simulated reflectance value to calculate a % error value, wherein the initial simulated reflectance value is generated by initializing a multi-layer Monte Carlo Simulation program with homogenous tissue optical properties across multiple layers of skin to simulate diffuse light reflectance for a lighter skin tone;
(4) perturbing the multi-layer Monte Carlo Simulation program by adjusting the light absorption coefficient for one of the layers of skin, while keeping the light absorption coefficients for the other layers of skin constant, and keeping the light scattering coefficients for the plurality of layers constant, to generate a revised simulated reflectance value;
(5) comparing the light reflectance values of step (2) with the simulated reflectance value of step (3) and the revised simulated reflectance value of step (4) to calculate a % error value, wherein if the calculated % error value meets a minimum % error value, the obtained light reflectance value is considered to be an accurate light reflectance value, and one can proceed to step (6), otherwise repeat steps (4) and (5); and
(6) quantifying the optical properties of the subject's skin at each wavelength based upon the inputs of the simulation required to model the accurate light reflectance value.
2. The multilayered, iterative method of aspect 1, wherein the light projected onto the portion of the subject's skin has wavelengths from 100 nm to 2000 nm, particularly, from 350 nm to 1000 nm, more particularly, from 500 nm to 850 nm.
3. The multilayered, iterative method of aspect 1 or aspect 2, wherein the light projected onto the portion of the subject's skin comprises 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 520, 540, 560, 570, 580, 600, 650, 700, 750, 800, 850, 900, 950, 1000, 1100, 1200, 1300, 1400, 1500, 1600, 1700, 1800, 1900, 2000, 2500, 3000, 3500, 4000, 45000 or 5000 different wavelengths, or a range that includes or is between any two of the foregoing numbers, particularly, from 2 to 4000 different wavelengths, more particularly, from 2 to 2000 different wavelengths.
4. The multilayered, iterative method of any one of the preceding aspects, wherein the light projected onto the portion of the subject's skin comprises 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 520, 540, 560, 570, 580, 600, 650, 700, 750, 800, 850, 900, 950, 1000, 1100, 1200, 1300, 1400, 1500, 1600, 1700, 1800, 1900, 2000, 2500, 3000, 3500, 4000, 45000 or 5000 different spatial frequencies, or a range that includes or is between any two of the foregoing numbers, particularly, from 2 to 300 different spatial frequencies, more particularly, from 5 to 100 different spatial frequencies.
5. The multilayered, iterative method of any one of the preceding aspects, wherein the imaging or light sensing device or system is selected from Spatial Frequency Domain Imaging, Temporal Frequency Domain Diffuse Optical Spectroscopy, Spatial Frequency Domain Spectroscopy, Time-Resolved Diffuse Optical Spectroscopy, Spatially-Resolved Diffuse Optical Spectroscopy, Continuous-wave near-infrared spectroscopy, Optical Coherence Tomography, pulse oximetry, dermoscopy, and colorimetry.
6. The multilayered, iterative method of aspect 5, wherein the imaging or light sensing device or system is Spatial Frequency Domain Imaging.
7. The multilayered, iterative method of any one of the preceding aspects, wherein the subject has a skin tone that is graded type I, type II, type III, type IV, type V, or type VI on the Fitzpatrick scale.
8. The multilayered, iterative method of aspect 7, wherein the subject has a skin tone that is graded type IV, type V, or type VI on the Fitzpatrick scale, particularly, wherein the subject has a skin tone that is graded type V or type VI on the Fitzpatrick scale
9. The multilayered, iterative method of any one of the preceding aspects, wherein the accurate light reflectance value correlates with the concentration of a major chromophore found in the subject's skin.
10. The multilayered, iterative method of aspect 9, wherein the major chromophore is selected from oxyhemoglobin, deoxyhemoglobin, and melanin.
11. The multilayered, iterative method of aspect 10, wherein the major chromophore is melanin.
12. The multilayered, iterative method of any one of the preceding aspects, wherein the multiple layers of skin comprise 2 layers of skin: a top epidermis layer, and a bottom dermis/hypodermis layer.
13. The multilayered, iterative method of aspect 12, wherein the thickness of the top epidermis layer is set a fixed value of 10 nm, 15 nm, 20 nm, 25 nm, 30 nm, 35 nm, 40 nm, 45 nm, 50 nm, 55 nm, 60 nm, 70 nm, 75 nm, 80 nm, 85 nm, 90 nm, 95 nm, 100 nm, 150 nm, 200 nm, 250 nm, 300 nm, 350 nm, 400 nm, 450 nm, 500 nm, 600 nm, 700 nm, 800 nm, 900 nm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1 mm, 1.1 mm, 1.2 mm, 1.3 mm, 1.4 mm, or 1.5 mm, or a range that includes or is between any two of the numbers, particularly from 10 nm to 500 μm, more particularly, from 30 nm to 300 μm.
14. The multilayered, iterative method of aspect 12, wherein the light absorption coefficient for the epidermis layer is perturbed in the Monte Carlo Model.
15. The multilayered, iterative method of any one of the preceding aspects, wherein the multi-layer Monte Carlo Simulation is a 2-layered Monte Carlo Simulation.
16. The multilayered, iterative method of any one of the preceding aspects, wherein the optical properties of the skin being measured are selected from tissue absorption coefficient, tissue reduced scattering coefficient, and/or tissue oxygenation (StO₂).
17. The multilayered, iterative method of any one of the preceding aspects, wherein the portion of the subject's skin comprises a skin lesion selected from a skin cancer, an ulcer, or a burn wound; and wherein the multilayered, iterative method quantifies the optical properties of the skin lesion.
18. The multilayered, iterative method of claim 17, wherein the subject has a skin tone that is graded type I, type II, type III, type IV, type V, or type VI on the Fitzpatrick scale, particularly, wherein the subject has a skin tone that is graded type IV, type V, or type VI on the Fitzpatrick scale, more particularly, wherein the subject has a skin tone that is graded type V or type VI on the Fitzpatrick scale

The following examples are intended to illustrate but not limit the disclosure. While they are typical of those that might be used, other procedures known to those skilled in the art may alternatively be used.

### EXAMPLES

Spatial Frequency Domain Imaging (SFDI). The Oxlmager RS™ (Modulim, Inc, Irvine, Calif.) was used to acquire and analyze measurements of ventral forearm data from 8 subjects having different skin tones. The system measures tissue reflectance over a wide range of view (20 cm×15 cm). LEDs at 8 wavelengths from 471 nm to 850 nm will project sinusoid pattern of 5 spatial frequencies from 0 mm⁻¹ to 0.2 mm⁻¹. Typically, the reflectance images are calibrated against a silicon phantom of know optical properties, and used, in combination with a multi-frequency look-up-table, to calculate the absorption coefficient and scattering coefficient. This analysis is completed within the accompanying MI Analysis software suite (Modulim Inc., Irvine, Calif.). Further analysis of was performed in MATLAB(R2020a, Mathworks, Natick, Mass.). A 40×40 pixel region of interest was chosen from the ventral forearm image for further analysis.

Subjects. Subjects were classified into 2 groups based on their Fitzpatrick graded skin tone (e.g., see FIG. 2) evaluated from clinician. Group I consisted of 3 subjects having Fitzpatrick Type I, II and III graded skin tones ; Group II consists of 5 subjects having Fitzpatrick type IV, V and VI graded skin tones. An additional patient, having a Fitzpatrick II graded skin tone and a high L* (66.4), was used as a reference for optical property values, as these measurements would be the least confounded by the presence of epidermal melanin.

Colorimetry. The L* value used in this study was obtained from a skin colorimeter in CIE L*a*b* color space (Chroma Meter CR-400, Konica Minolta Sensing, Inc., Tokyo, Japan). The colorimeter provided a more quantitative measurements of skin darkness compared to the class-based Fitzpatrick scale. L* parameter is used to represent skin “lightness” on a 0-100 scale (0=darkest, 100=lightest).

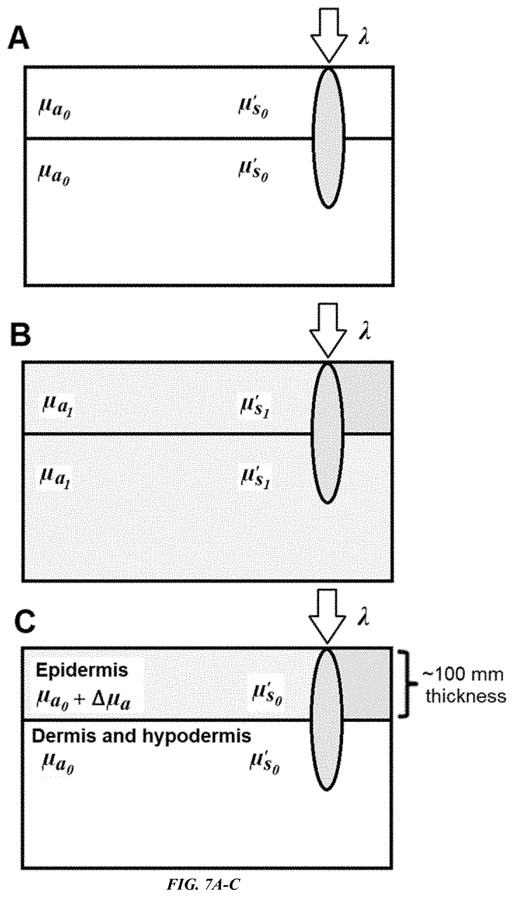

Homogeneous Skin Model. The first analysis technique used was based on a homogeneous model, which treats both the epidermis and dermis of the skin as having the same optical properties. This model is shown in FIG. 7A-B. While this method is typical for SFDI measurements, this model is not physiologically accurate when describing patients having darker skin tones. Most melanin is distributed in the epidermis of the skin, as shown in FIG. 7C and FIG. 8. Therefore, a layered model is more physiologically relevant to real skin structure.

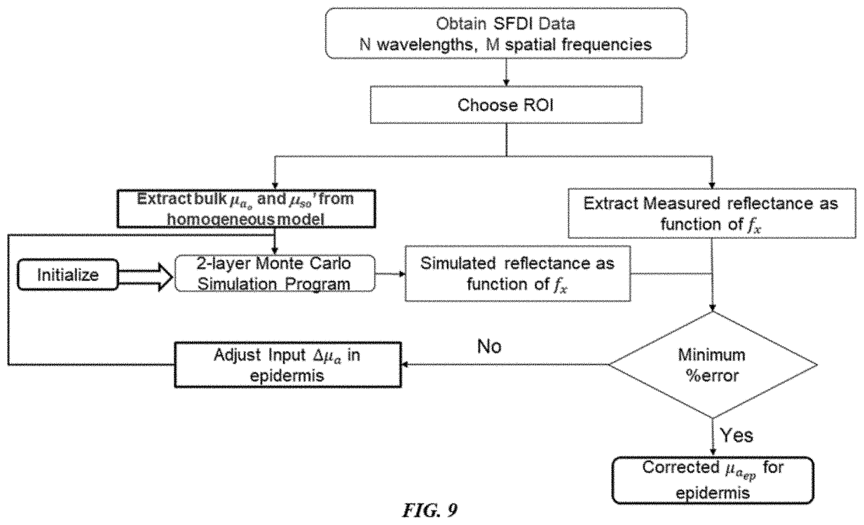

Multilayer Model Fitting Procedures. Melanin has a very strong absorption in the visible range and is distributed in the epidermis of the skin. Therefore, the first perturbation in the layered model changes only μₐ in the first layer. It was also assumed that the rest of the tissue optical properties (μₐ, μₛ of dermis and μₛ of epidermis) to be constant in this case. These properties are set to be the same values as the light skin tone subject. For the input geometry of the Monte Carlo Simulations, the thickness of epidermis was set to be 100 microns. The number of photons is 100k for accurately simulating the diffuse reflectance. After initialization, the parameters were inputted into a 2-layer model, and the simulated reflectance was calculated and compared with the measured reflectance. The relative differences between the measured data and the model were calculated in order to optimize the input parameters. δμₐ was adjusted after the first iteration, and the perturbation process was continued until a minimum difference was attained. A detailed flowchart of the foregoing process is shown in FIG. 9.

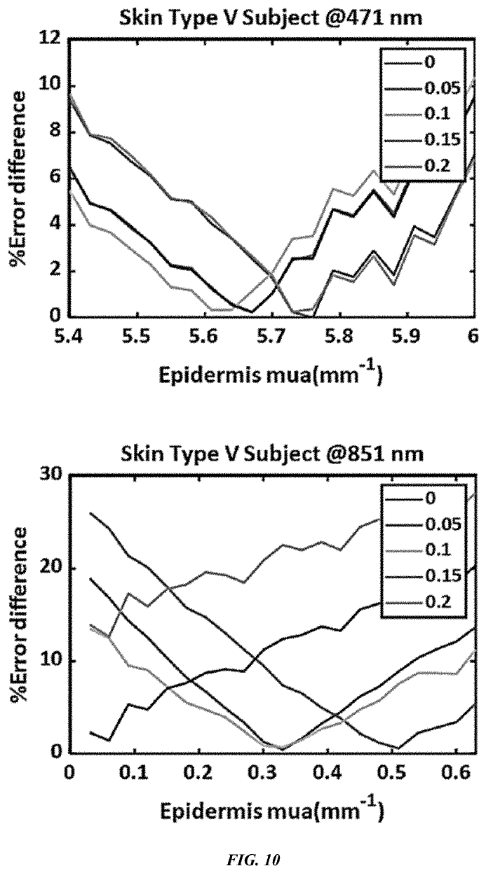

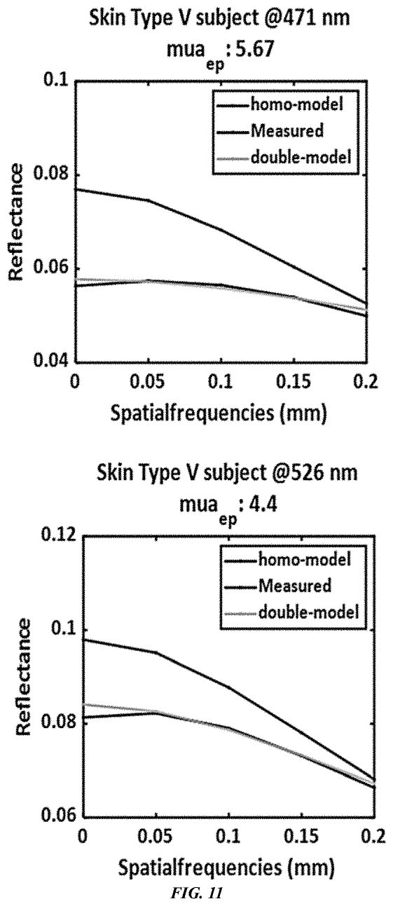

Comparison between fitting using homogeneous model and layered model. A preliminary test presented in FIG. 10 shows that as the epidermal μₐ increases, a local minimum in the difference between simulated reflectance and measured reflectance can be located. This means that an optimal epidermal μₐ can be extracted from the iterative method. Since the simulation program returns the reflectance spectra of 5 spatial frequencies, the local minima from the different spatial frequencies are not the same. Here, the best epidermal μₐ was extracted by selecting the minimum value after averaging the differences between the measured and modeled data for all 5 spatial frequencies.

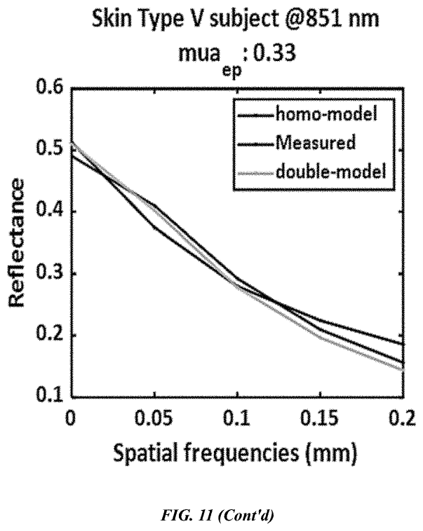

Once the best-fit epidermal μₐ is extracted, the results of the layered model simulation were compared with a homogeneous model simulation. FIG. 11 shows the reflectance spectrum comparison between the homogeneous (labeled “homo-model”) and the layered model (labeled “double model”). At shorter wavelengths, the results of the 2-layered simulation compared favorably against the actual measured reflectance, unlike the homogeneous model. At the longer wavelengths, both different models perform similarly. This is somewhat expected, as melanin has a lower absorption at these wavelengths.

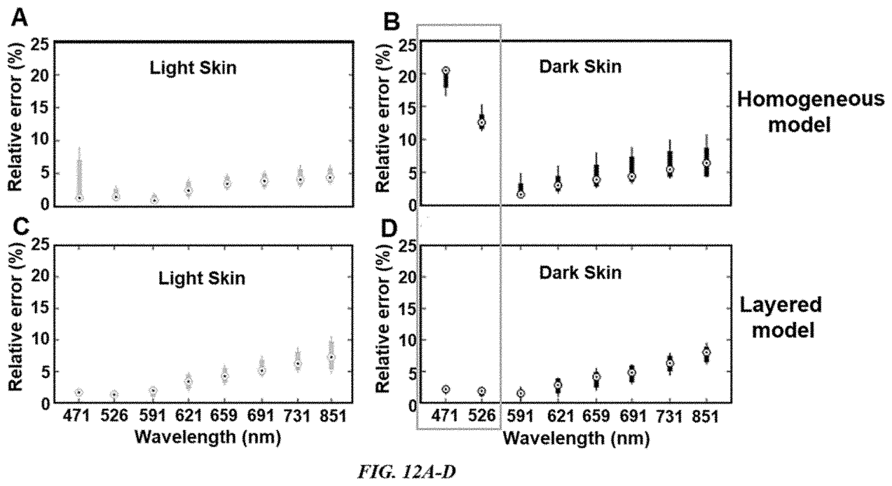

This same process was applied for all subjects in the study, FIG. 12A-D shows the boxplot of different models among different skin tones. In FIG. 12A and C, detailing both model outputs for the low Fitzpatrick score Group 1 subjects, the layered model has similar relative fitting errors compared to the homogeneous model. However, in FIG. 12B and D, for subjects having greater pigmentation in Group 2, the layered model has a much lower relative error compared to the homogeneous model at shorter wavelengths.

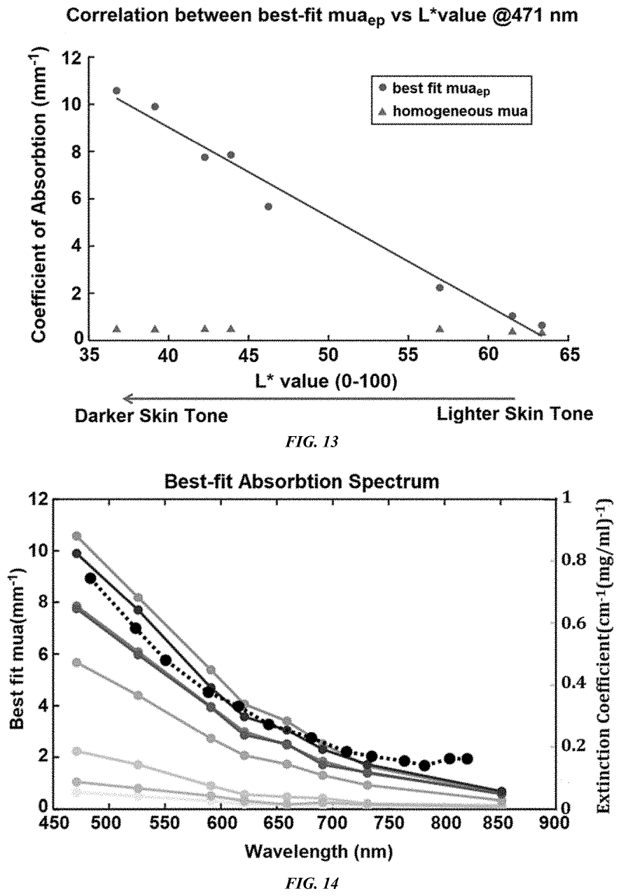

Analysis of extracted μₐ. In FIG. 13, looking at the epidermal μₐ, the inverse fitting procedure was realized, it is much higher than the homogeneous μₐ generated from the device, especially for the subjects having lower L* values, or high pigmentation. Correlation analysis shows a strong linear relationship between best-fit epidermis μₐ and the L* measured by a chromameter. It can be concluded that the epidermal μₐ directly corresponds to skin pigmentation, and correlates with the μₐ due to melanin.

FIG. 14 presents the best fit melanin μₐ spectrum of all 8 subjects. The darker color curves represent subjects having darker skin tones. Curves follow exponential decay functions. By fitting the curves to a standard eumelanin extinction coefficient spectrum curve (black dotted line), the concentration of melanin can be calculated.

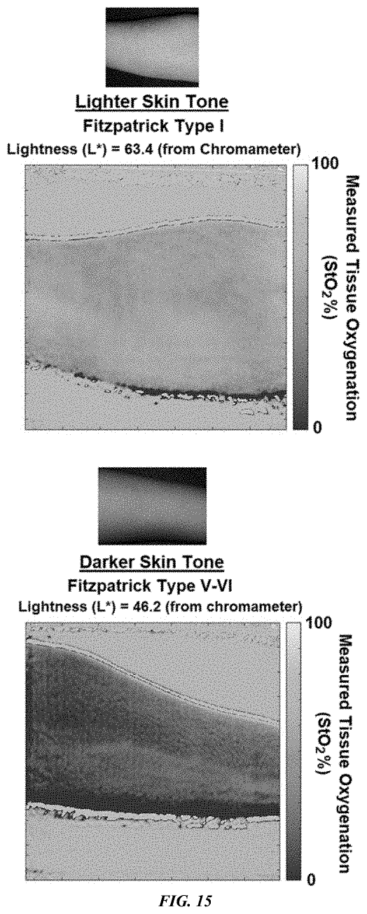

Effects of skin tone on measured tissue oxygenation (StO₂) values using SFDI. FIG. 15 shows noticeable differences in measured StO₂ from a healthy subject with a lighter skin tone (left) v. a healthy subject with a darker skin tone (right) using a SFDI device in a homogeneous tissue model. While the measured StO₂ from the subject with a lighter skin tone was within a range typical of SFDI technology, the measured StO₂ from the subject with a darker skin tone was not a physically realistic value.

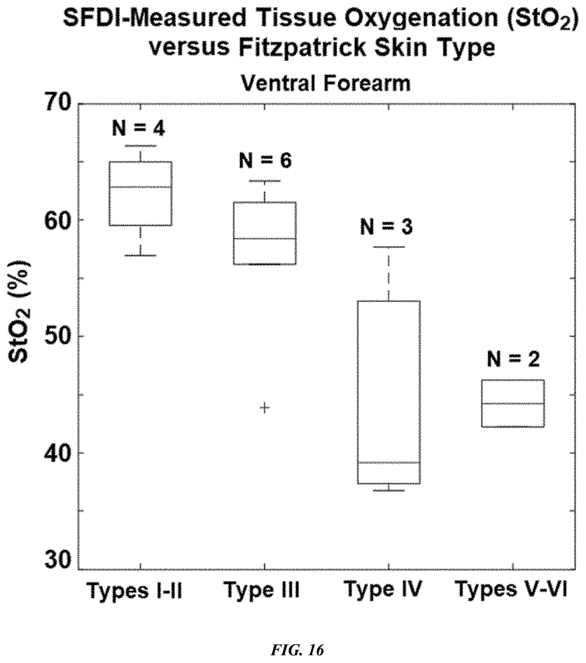

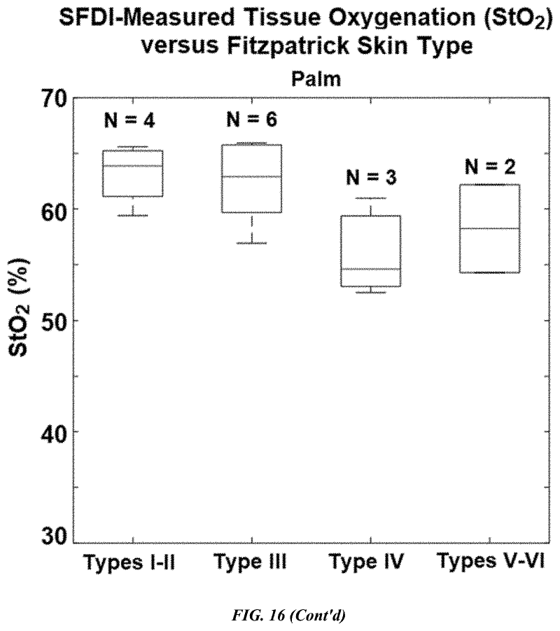

Next, the relationship between Fitzpatrick skin type and measured StO₂ using a commercial SFDI device with a homogeneous tissue model was evaluated. For the palm, which has much less melanin than the ventral forearm, subjects of all skin tones have similar StO₂ values (e.g., see FIG. 16). For the ventral forearm, subjects having lighter skin (Types I-III) have similar measured StO₂ values, but subjects having darker skin (Types IV-VI) have systematically much lower measured StO₂ values (see FIG. 16). The data suggests that that for tissues where melanin is not present, baseline StO₂ measurements are similar for all subjects, but for tissues where melanin is present, StO₂ measurements can be significantly confounded by melanin. Since tissue oxygenation should not be affected by skin color, these results illustrate an inaccuracy of standard homogeneous models for tissue oximetry using diffuse optics technology.

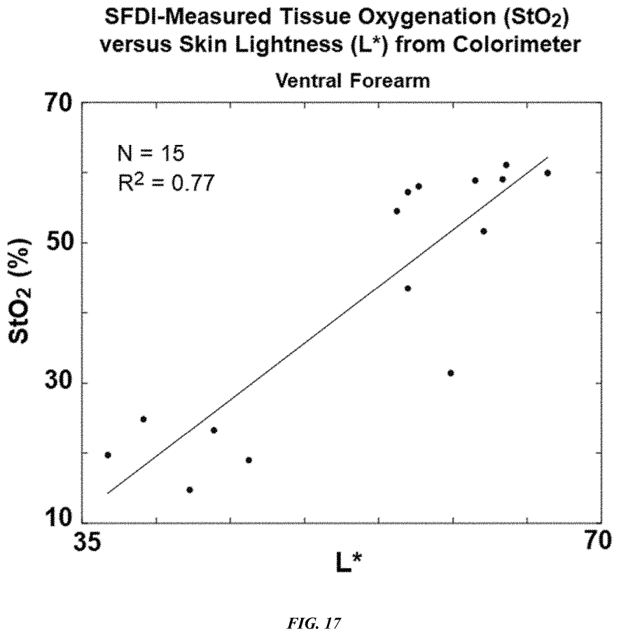

Evaluating the relationship between skin “lightness” parameter (L*), measured via colorimetry, and measured StO₂ using an SFDI device in a homogeneous tissue model. Utilizing ventral forearm measurements on 15 healthy subjects of wide-ranging skin colors, it was found a strong linear correlation between the measured values of L* and StO₂: subjects having lighter skin (higher L*) have systematically higher measured values of StO₂, even though there is no reason to expect that tissue oxygenation should be physiologically related to skin tone (see FIG. 17). Therefore, the results indicate a significant bias in accuracy of the commercial SFDI device toward patients with lighter skin tones when the homogeneous tissue model is used.

A number of embodiments have been described herein. Nevertheless, it will be understood that various modifications may be made without departing from the spirit and scope of this disclosure. Accordingly, other embodiments are within the scope of the following claims.

What is claimed is:

1. A multilayered, iterative method to accurately quantify optical properties of skin in subjects that have a wide range of skin tones, comprising:
(1) projecting light comprising a plurality of wavelengths and/or spatial frequencies onto a portion of a subject's skin;
(2) obtaining light reflectance values from the subject's skin using an imaging or light sensing device;
(3) comparing the obtained light reflectance values for the plurality of wavelengths and/or spatial frequencies with an initial simulated reflectance value to calculate a % error value, wherein the initial simulated reflectance value is generated by initializing a multi-layer Monte Carlo Simulation program with homogenous tissue optical properties across multiple layers of skin to simulate diffuse light reflectance for a lighter skin tone;
(4) perturbing the multi-layer Monte Carlo Simulation program by adjusting the light absorption coefficient for one of the layers of skin, while keeping the light absorption coefficients for the other layers of skin constant, and keeping the light scattering coefficients for the plurality of layers constant, to generate a revised simulated reflectance value;
(5) comparing the light reflectance values of step (2) with the simulated reflectance value of step (3) and the revised simulated reflectance value of step (4) to calculate a % error value, wherein if the calculated % error value meets a minimum % error value, the obtained light reflectance value is considered to be an accurate light reflectance value, and one can proceed to step (6), otherwise repeat steps (4) and (5); and
(6) quantifying the optical properties of the subject's skin at each wavelength based upon the inputs of the simulation required to model the accurate light reflectance value.

2. The multilayered, iterative method of claim 1, wherein the light projected onto the portion of the subject's skin comprises wavelengths from 100 nm to 2000 nm.

3. The multilayered, iterative method of claim 1, wherein the light projected onto the portion of the subject's skin comprises 2 to 2000 different wavelengths.

4. The multilayered, iterative method of claim 1, wherein the light projected onto the portion of the subject's skin comprises 2 to 3000 different spatial frequencies.

5. The multilayered, iterative method of claim 1, wherein the imaging or light sensing device or system is selected from Spatial Frequency Domain Imaging, Temporal Frequency Domain Diffuse Optical Spectroscopy, Spatial Frequency Domain Spectroscopy, Time-Resolved Diffuse Optical Spectroscopy, Spatially-Resolved Diffuse Optical Spectroscopy, Continuous-wave near-infrared spectroscopy, Optical Coherence Tomography, pulse oximetry, dermoscopy, and colorimetry.

6. The multilayered, iterative method of claim 5, wherein the imaging or light sensing device or system is Spatial Frequency Domain Imaging.

7. The multilayered, iterative method of claim 1, wherein the subject has a skin tone that is graded type I, type II, type III, type IV, type V, or type VI on the Fitzpatrick scale.

8. The multilayered, iterative method of claim 7, wherein the subject has a skin tone that is graded type IV, type V, or type VI on the Fitzpatrick scale.

9. The multilayered, iterative method of claim 1, wherein the accurate light reflectance value correlates with the concentration of a major chromophore found in the subject's skin.

10. The multilayered, iterative method of claim 9, wherein the major chromophore is selected from oxyhemoglobin, deoxyhemoglobin, and melanin.

11. The multilayered, iterative method of claim 10, wherein the major chromophore is melanin.

12. The multilayered, iterative method of claim 1, wherein the multiple layers of skin comprise 2 layers of skin: a top epidermis layer, and a bottom dermis/hypodermis layer.

13. The multilayered, iterative method of claim 12, wherein the thickness of the top epidermis layer is set a fixed value from 30 nm to 300 μm.

14. The multilayered, iterative method of claim 12, wherein the light absorption coefficient for the top epidermis layer is perturbed in the Monte Carlo Simulation program.

15. The multilayered, iterative method of claim 1, wherein the multi-layer Monte Carlo Simulation is a 2-layered Monte Carlo Simulation.

16. The multilayered, iterative method of claim 1, wherein the optical properties of the skin being measured are selected from tissue absorption coefficient, tissue reduced scattering coefficient, and/or tissue oxygenation (StO₂).

17. The multilayered, iterative method of claim 1, wherein the portion of the subject's skin comprises a skin lesion selected from a skin cancer, an ulcer, or a bum wound; and wherein the multilayered, iterative method quantifies the optical properties of the skin lesion.
